(19) United States
(12) Patent Application Publication
(10) Pub. No.: US 2006/0040330 A1
(43) Pub. Date: Feb. 23, 2006

(54) Method of cancer screening

(76) Inventors: Igor Ivakhnenko, Silverspring, MD (US); Alexander Balyura, Moscow (RU)

Correspondence Address: ROSENBERG, KLEIN & LEE, 3458 ELLICOTT CENTER DRIVE-SUITE 101, ELLICOTT CITY, MD, 21043, US

(21) Appl. No.: 11/138,334

(22) Filed: May 27, 2005

(30) Foreign Application Priority Data
Aug. 23, 2004 (RU) 2004125522

(51) Int. Cl. G01N 33/574; C12Q 1/56

(52) U.S. Cl. 435/7.23; 435/13

(57) ABSTRACT

Erythrocyte sedimentation rate (“ESR”) determinations of a patient's blood that has been combined either with blood serum from pregnant mammals or with fetal embryonic serum yield measurable results correlating well with the presence of an on-going cancer. This cancer detection method based on differential ESR determinations consists of paired in vitro ESR tests, separately combining a patient's whole blood with a control serum and with a test serum comprising either fetal embryonic serum or serum from a pregnant mammal, to give a cancer coefficient value that correlates with a probability of an on-going malignancy. The probability of an on-going malignancy is determined by reference to historical data correlating the cancer coefficient and the presence of ongoing cancer. A kit for performing the cancer screening methodology is also provided.

### PRIORITY REFERENCES

This Patent Application claims priority based on Russian Patent Number 2004125522, issued 23 Aug. 2004.

### FIELD OF THE INVENTION

The subject Patent is directed to medical and veterinary diagnostic tests, particularly to cancer screening laboratory tests, and even more particularly, to immunologic tests for the presence of neoplastic diseases in humans and other mammals. The present invention directs itself to an in vitro medical cancer screening test that compares erythrocyte sedimentation rates of a test subject's blood mixed with an aliquot of embryonic fetal serum, and in the alternative an aliquot of serum from a pregnant mammal, with a control erythrocyte sedimentation rate test to establish the probability of the patient having cancer. The present invention is further directed to a new use for erythrocyte sedimentation rate determinations, as well as to new uses for fetal embryonic serum and for serum from pregnant mammals. Additionally, the subject invention is directed to a cost-effective oncology screening protocol comprising an in-vitro methodology using the serum of pregnant mammals and alternatively, fetal embryonic serum to screen test subjects for on-going neoplastic processes.

### BACKGROUND OF THE INVENTION

There exist numerous tests to screen patients for cancer, most of them concerned with screening for a particular type of cancer. There are many in vitro tests used to indicate the presence of a malignant neoplastic process within a patient but these all have in common problems of poor specificity and/or sensitivity and/or predictive value.

Early diagnosis of cancer is extremely important. The outcome of cancer treatments depends on early detection. Thus, a reliable test for early cancer detection would help enormously since the survival rate with modern cancer therapies in cases of early detection is higher than 90%. Presently, many cases of cancer go unnoticed until severe symptoms are manifested, at which late stages of cancer too many cases are refractory to present treatments.

Since its introduction in the early 1920's, the erythrocyte sedimentation rate (“ESR”) has been used in various ways for such screening of patients with suspected malignancies. A more reliable and straightforward use of the ESR for cancer screening has been frustrated by the ESR test's lack of predictability and specificity, and by its excessive sensitivity to both malignant and non-malignant pathologies. On average, the ESR will miss 25% of those with established neoplastic malignant disease diagnoses—so-called ‘false negatives’—and give a positive, which is to say elevated reading in numerous non-cancer inflammatory conditions, so-called ‘false positives’.

The ESR is a simple and inexpensive laboratory test using the whole blood of a patient or mammal. The collected blood is anti-coagulated and then introduced into standardized capillary tubes for ESR determination. ESR measures the volume of plasma above the column of settled blood cells after the red blood cells have been allowed to settle for a predetermined amount of time, typically an hour or less an hour in a properly calibrated tube. Certain technical factors in the ESR effect the aggregation of red blood cells to rouleaux or stacked formations, including the particular characteristics of the erythrocytes such as size, shape and surface charge; the viscosity of the plasma; and the interacting electrostatic forces of the surrounding macromolecules, notably fibrinogen, albumen and gamma globulins.

Erythrocytes normally repel each other as a result of the negative charges of the carboxyl group of N-acetylneuraminic acid located on the surface of the red blood cell. Aggregation of erythrocytes is increased when these negative charges are neutralized to a greater or lesser extent by circulating plasma macromolecules, thereby promoting rouleaux formation as well as adding weight to the red blood cell by the adhesion of these circulating plasma macromolecules. The result is an increased rate at which the erythrocytes fall to the bottom of the capillary tube used in ESR determination techniques, hence an increased ESR value.

ESR determinations are quite sensitive to extrinsic factors, such as room temperature, the amount of anticoagulant added to the specimen to prevent clot formation, the length of time between sample collection and testing, the deviation of the test tubes from a strictly upright position, the presence of any bubbles in the test tube, and the length of time at which the ESR value is established. These extrinsic factors can be controlled to a great extent by following consistent procedural standards.

The present subject invention encompasses in a preferred embodiment the use of tilted capillary tubes during the erythrocyte sedimentation phases in the ESR methodology, tilting away from the vertical at an angle between about 10° and about 55°, preferably at 45°. The inventors have surprisingly discovered that the rate of ESR equilibration, that is to say the time until the ESR test reaches a plateau in erythrocyte rouleaux formation and settling, is roughly halved when the capillary tubes are tilted at a 45° angle.

Without intending in any way to be bound by theory, it is believed that the adhesion of gamma globulins to erythrocytes occurs primarily at surface layers where the force of surface tension accelerates the molecular interactions. When a capillary tube is tilted away from the vertical at 45°, the surface area doubles. This correlates well with the inventors' observation that ESR determination tests can be done in roughly half the time when tilted 45° compared with the customary upright position without any degradation of specificity, sensitivity, predictability or reliability.

Currently, ESR determinations are found useful to obtain three types of information: 1, to determine the presence or absence of disease; 2, to monitor the progression or improvement of an already diagnosed disease; and 3, to measure the response to treatments. Although the present invention is described for diagnostic purposes to determine the presence or absence of a neoplastic disease process in a patient, the other two related uses of the subject methodology are within the scope and contemplation of this inventive concept.

The significance of a diagnostic test, its utility, is reflected by the test's sensitivity, specificity, and positive predictive value. The number of patients who test positive and who also have a neoplastic disease is designated ‘A’; those who test positive but who do not have cancer are designated ‘B’; those who have a neoplastic disease but whose test results are negative are represented as ‘C’; and those with a negative test result who do not have a neoplastic disease are designated ‘D’. The quality and utility of the subject cancer screening methodology when practiced in one series of clinical investigations is summarized in Table 1, which relates to the clinical data presented in Table 2.

TABLE 1

| TEST RESULT ↓ | DISEASE PRESENT | DISEASE NOT PRESENT |
|---|---|---|
| Positive | A (n = 915) | B (n = 0) |
| Negative | C (n = 96) | D (n = 24) |

The sensitivity of a diagnostic test is defined as the probability that when the disease is present the test results will be positive; sensitivity of the test is calculated as A divided by the sum A+C. Sensitivity=A/(A+C)

The specificity of the test is defined as the probability that the test is negative when no disease is present; the specificity is thus calculated by dividing B by the sum B+D. Specificity=D/(B+D)

The positive predictive value of a test is the probability that a positive test result reflects the presence of disease. Positive Predictive Value=A/(A+B)

The data presented in Table 1 indicate for the differential-ESR diagnostic test values while practicing the preferred methodology of the present invention a test Sensitivity of 0.9, a test Specificity of 1.0, and a Predictive Value of 1.0, indicative of a significant degree of clinical usefulness for the subject inventive laboratory medical test methodology.

TABLE 2

| Cancer Diagnosis | # of Patients | Test Positive | Test Negative |
|---|---|---|---|
| Breast | 207 | 186 | 21 |
| Lung | 478 | 429 | 49 |
| Gastric | 95 | 87 | 8 |
| Enteric | 36 | 31 | 5 |
| Colon | 49 | 45 | 4 |
| Renal | 23 | 21 | 2 |
| Bladder | 41 | 38 | 3 |
| Gonadal | 22 | 20 | 2 |
| Thyroid | 15 | 14 | 1 |
| Leukemia | 1 | 1 | 0 |
| Prostate | 9 | 9 | 0 |
| Adenocarcinoma | 1 | 1 | 0 |
| Multiple Myeloma | 1 | 1 | 0 |
| Lymphosarcoma | 4 | 4 | 0 |
| Pancreatic | 1 | 1 | 0 |
| Hepatic | 5 | 5 | 0 |
| Lymphoma | 17 | 16 | 1 |
| Melanoma | 2 | 2 | 0 |
| Uterine | 3 | 3 | 0 |
| Salivary Glands | 1 | 1 | 0 |
| No Cancer (Healthy) | 24 | 0 | 24 |
| Total | 1011 | 915 | 96 |

ESR determination tests may be abnormally elevated as an indication of any of a diverse range of on-going diseases, which has led clinicians in the past to conclude that the ESR determination test was at best a marginally useful diagnostic test. Nonetheless, the simplicity and low cost of ESR determination testing have prompted numerous investigators to try to adapt the ESR for diagnostic applications in ways that improved the test's sensitivity, specificity and predictive value.

Very often the virulence, the relative malignancy of a particular tumor is not very evident. For example, about one out of ten cases of breast cancer is particularly dangerous, referred to often as a high grade malignancy associated with an anaplastic histology and poor prognosis; there is no current technology on the market to differentiate which cases are especially dangerous and which are not. It is common practice for doctors to recommend mastectomy if any doubt exists. If a cancer test existed which could give information as to what level or grade of the malignancy is present, many patients might be spared a mastectomy, which might not be indicated in a substantial number of cases.

The timely monitoring the effectiveness of cancer treatments is also an important goal of the subject invention. For example, the size and extent of a cancer may not be completely determinable prior to a surgical effort to remove all tumor. Complete removal of all tumor is associated with the differential ESR-based cancer coefficient of the present subject methodology reverting to a negative result within about two weeks of complete tumor removal. Whether the present screening test remains positive or reverts to negative can be an important factor in decisions regarding post-operative radiation and/or chemotherapy. A reliable timely cancer test could serve for such applications rather well.

There are many new medicines for treating cancers and it takes much time and money to determine the true effectiveness of these medicines. The subject inventive cancer screening test takes as another important goal to shorten the period of drug trials. Relatedly, the many new products and medicines have to be tested for any carcinogenic potential, a long and costly process. It is further a goal of the present subject invention to help shorten and economize carcinogenicity screening trials for drugs and chemical products.

It is the Inventors' belief that the subject diagnostic methodology solves these problems for cancer screening by adapting the ESR technique to their methodology with a differential ESR technique to yield a meaningful, reliable and cost-effective process for detecting the presence of a neoplasm in a patient.

An important concept of the subject invention is that blood serum taken from pregnant mammals or embryos and combined with whole blood of a patient can yield a measurable result—a cancer coefficient—using the ESR methodology combined with an immunologic reaction. The differential ESR technique compares the ESR of a patient's whole blood when combined with blood serum of a pregnant mammal or fetal embryonic serum, with the ESR of a patient's blood when combined with control serum from a non-pregnant mammal. The same inventive concept applies to tissue suspensions and other bodily fluids but the immunologic reactions associated with an on-going neoplastic disease must be detected by other methods, such as immunofluorescence.

In order to improve the accuracy of resulting determinations, an extended differential type of reaction is employed. For example, two different samples of patient blood are obtained and then two types of mammal serum are added to the two different samples: test serum obtained from a pregnant mammal is combined with a patient's first blood sample, and control blood serum taken from a non-pregnant mammal of the same species is added to a second blood sample of the same patient.

If the difference in reactions in two or more samples, the extended differential ESR value that is directly reflected in the cancer coefficient reaches a predetermined threshold value for the cancer coefficient, typically greater than 1.5 in the preferred embodiment, then the subject screening methodology is positive for an on-going malignancy. The cancer coefficient value is directly proportional to the ESR difference in millimeters mathematically weighted (by multiplication) with the maximal ESR measure and normalized to discount the various particular capillary tube dimensions.

In the preferred embodiment plastic SEDIPLAST® 100 mm capillary tubes (Polymedco, Cortland, N.Y.) are employed; the normalization factor has been determined heuristically to be division by 230. Also in the preferred embodiment, the predetermined difference in ESR reactions between the patient's serum and a test serum expressed as the cancer coefficient value is deemed positive if greater than 1.5.

The subject inventive method of malignant tumor screening avoids the difficulties of prior art screening methods by adaptively employing a commercially available source of embryonic fetal serum or its equivalent, such as serum from pregnant mares. The test serum, in this case the embryonic serum, can be produced in sufficiently large quantities and thereby transform a cumbersome and labor intensive laboratory effort into a practical clinical screening test. Control sera are commercially available and comprise serum made for other purposes, such as anti-tetanus serum, as long as the control serums are from non-pregnant mammals of the same species as the respective test serums.

### SUMMARY OF THE INVENTION

The subject invention's method of cancer screening for humans and other mammals preferably comprises these steps: a predetermined amount of an fetal embryonic or pregnant animal serum is combined with a sample of a patient's whole blood, and for the test's control, blood serum from a healthy non-pregnant mammal is added to another aliquot of the patient's whole blood.

The ESR value is established for each of the two blood samples tested. The difference between the two ESR determinations results allows the patient-specific establishment of a cancer coefficient, along with the concomitant probabilities of an on-going malignant neoplastic process: the likelihood of an on-going neoplastic pathology is a probability value between zero and one, assigned by using the cancer coefficient and nomograms derived from historical data matching cancer status with measured cancer coefficient.

Serum that is obtained from the blood of pregnant mares may be taken anytime during pregnancy but preferably during the second trimester, and more preferably between around the 45^{th} and 100^{th} day of gestation. The pregnant horse serum is then combined with an aliquot of a patient's blood and an ESR determination made. Then the differential ESR determination test of the present invention allows the quantification of the probability of an on-going cancer according to the difference in the ESR determinations between testing and control results.

Blood serum from various pregnant mammals, including without limitation horses, cows, sheep, goats, birds and so on, can be used as the test material for the present invention test. Alternatively, embryonic fetal serum can also be used as the test material for the screening test. The use of different pregnant mammals' blood serum and of different embryonic serum provides substantially independent conditions for the inventive screening test; it has been found that the more extensive testing, which includes the separate use of different sera at the same time, further improves the sensitivity and predictability of the invented test.

The in vitro method of cancer screening of the subject invention is not apparently sensitive to a malignant neoplasm's anatomic location or its particular histology. Conversely, the subject inventive screening test provides no information regarding the specific tumor histology or anatomic location.

Prior art methods of producing suitable test serum are extremely complicated as they require the double immunization of small syngeneic laboratory mammalian animals. Furthermore, the use of small laboratory mammals does not support the commercially practicable production of substantial amounts of fetal embryonic test serum. Additionally, these same prior art methods are extremely difficult to apply to large mammals such as, for example, a horse. For example, embryonic tissue would be implanted into a horse, and then the horse spleen would be surgically removed with the splenic lymphocytes harvested then injected to the same genotype horse, and so on. It is obvious that such methods would be impractical.

The inventive methodology provides for a pregnant mammal's blood serum to be used as a test serum and contrasted with a control serum to determine vel non a presumptive cancer diagnosis. For example, when blood serum of pregnant mares is used as a test serum; blood serum from a normal non-pregnant horse is used as a control serum.

It is the Inventors' belief that the theoretical basis for this novel cancer screening test suitable for mass production is the paraparthenogenetic hypothesis of oncogenesis, which was proposed by Dr. Alexander Balyura in 1983. According to this hypothesis, malignant changes happening in a differentiating somatic cells cause paraparthenogenetic activation of a cellular genetic mechanism, which switches on two successive genetic programs: program of a normal development of a somatic cell blocked at some stage of differentiation and a program of normal oncogenesis which starts from the very beginning. The last program of normal oncogenesis starts only for some cells.

This theory is supported by a number of experimental facts. For example, it has been found that malignant tumors consist of two different kinds of cells: one kind are similar to cells of normal tissue from which the tumor originated (tumor stem cells), and the second kind (differentiating from tumor stem cells) are similar to normal cells from which the tumor originated but which with time become more and more similar to the differentiated cells of other organs and tissue. Rates of such progression will be different and highest for poorly differentiated (anaplastic) tumors, medium—for moderately differentiated tumors, and low—for highly differentiated tumors. It is the Inventors' opinion that all carcinogenic substances, including chemical agents and viruses, trigger this paraparthenogenetic mechanism, which is the same for all tumors; for some reason oncogenesis does not start in non-malignant tumors.

The following evidence favors this paraparthenogenetic hypothesis:
1) It was shown experimentally that Teratocarcinoma of the testicle in humans and animals consists of a mixture of cell types: one type is similar to testicular cells of an embryo, and other type have similarities to 14 types of cells different organs and tissue. It was demonstrated that if one cell of the first type is implanted it triggers growth of the same teratocarcinoma, and if other type cell is implanted it does not trigger tumor growth;
2) Expression of oncogenes (c-srk, c-myc, c-erb, crash, c-resk, c-sis, N-myc, L-myc) has been demonstrated with tumor growth and with the early stages of embryogenesis. It seems that oncogenes are normal components of embryogenesis;
3) Messenger RNA (mRNA) found in a cancer cell cytoplasm is very similar to embryonic mRNA at early stages of growth;
4) Cancer cells generate products which typically are found in embryonic cells during early stages of growth. For a human embryo this period starts at gametogenesis and continues up to 10 weeks of growth. When genesis of organs is finished, generation of at least some products common for embryo cells and for cancer cells stops also;
   A central tenet of the paraparthenogenetic hypothesis is that a key difference between normal cells and cancer cells is that cancer cells even at early stages (several cells) of tumor growth produce early embryonic antigens (antigens specific for a stage of growth).

Works of a number of authors have independently arrived at this conclusion. For example, F. Muller (1864) and E. Heckell (1866) formulated a basic bio-genetic law in accordance with which during development of each individual a history of genesis of all ancestors of this individual repeats in short and compressed issue. For example, it was demonstrated that during human embryogenesis with time progression such antigens appear as are typical for frog, snake, and only later for humans.

Many similarities have been found between malignant tumor cells and embryonic cells at early stages of development. More than 70 years ago Hirshfeld and Halber experimentally found that blood serum from rabbits immunized with rat embryo cells reacted positively in binding with lipoid extract from rat placental tissue and also with lipoid extract from Jensen sarcomas, and an even more positive binding reaction with lipoid extract from human cancer tissue. At the same time, this serum did not react with extracts from human normal tissue; this result has been confirmed.

It is believed that embryonic blood antigens, including early stages of genesis (early embryonic antigens, stage specific antigens), and which do not exist in mother blood, they can pass placenta and cause generation of specific antibodies in mother blood. Such specific antibodies are found in all cases normally progressing pregnancy, and according to some authors opinion they positively regulate and normalize growth of an embryo. However, there are cases when during seemingly normal pregnancy “immunologic conflict” occurs between mother and an embryo. It happens sometimes with pregnant horses. Especially often such cases occur if a mare is coupled with an ass and an embryo got antigens from an ass. These antigens immunizing mare generate specific antibodies, which kill an embryo. It is known that in southern regions of France death rate at coupling of mares and asses mounts up to 15%.

It has been theorized that antibody generation starts a chain of immunologic reactions. In 1974 Jerne published his concept of an idiotypical net, accordance to which the immune response to antigen are antibodies that may have autoimmunogenic idiotypical determinants. In other words, each antibody (At1) may induce the generation of anti-idiotypical antibodies (At2), which in their turn induce the production of anti-anti-idiotypical antibodies (At3), which in their turn may induce the generation of anti-anti-anti-idiotypical antibodies (At4), etc.

Anti-idiotypical antibodies (At2) and anti-anti-anti-idiotypical antibodies (At4) carry an “antigen internal design”, i.e. they have antigen determinants that can bind with At1 and At3.

It has been shown that antigens and idiotypes are expressed on antigen binding receptors of T and B-lymphocytes, and that they are expressed on the outer membranes of erythrocytes, and further that there are antigens and idiotypes in blood serum.

The inventors believe that the facts presented support the consideration that inside of a cancer patient early embryonic antigens (stage specific) are generated, which induce the generation antibodies (At1) to early embryonic antigens, which in their turn cause the generation of anti-idiotypical antibodies (At2), which in their turn cause the generation of anti-anti-idiotypical antibodies (At3), etc. These early embryonic antigens and At1, At2, At3, At4 . . . are expressed on T and B-lymphocytes, on erythrocytes and they are present in blood serum of cancer patients. Thus, the antigens and anti-idiotype anti-embryonic antibodies present in the test serums reacts with embryonic-type antigens found in a cancer patient's whole blood and affects the ESR reaction in a quantifiable manner.

### DETAILED DESCRIPTION OF THE PREFERRED EMBODIMENT

The patient to be diagnostically screened using the present subject cancer screening method is understood and defined herein to be a human or any other mammal. Venous, arterial or capillary whole blood can be used for the tests.

According to the subject invention, the presumptive cancer screening result, the cancer coefficient value, is obtained as the follows:

Fetal embryonic or pregnant animal serum is added to one sample or aliquot of a patient's whole blood and blood serum from a normal mammal of the same species is added to another aliquot of the patient's whole blood. Then, the Erythrocyte Sedimentation Rate (“ESR”) is determined for each mixed blood sample. The difference in ESR determinations is established and a cancer coefficient is calculated according to the heuristically derived formula of the present subject invention. Slightly different formulae can be used equivalently as long as the resulting values are normalized to allow statistical comparison with cancer coefficients correlated with known cancer cases.

The test serum used is blood serum from pregnant mares, preferably taken during the second trimester, and more preferably in the 45^{th} to 100^{th} day of gestation; serum from a non-pregnant horse is used as a control serum.

Likewise, when blood serum of other pregnant mammals, such as, without limitation, pregnant cows, dogs, sheep, goats, pigs and so on, is used as the test serum then serum from non-pregnant cows, dog, sheep, goats, pigs etc, respectively, is used as the control serum.

In a preferred embodiment, fetal embryonic blood serum is used as the test serum and normal serum of the same mammal species used as the control serum.

In other embodiments, blood serum of a horse, cow, dog, sheep, goat etc. embryo is used as the test serum and normal serum of the horse, cow, dog, sheep, goat etc respectively used as the control serum.

In order to improve the sensitivity and specificity of the test, preferred embodiments of the subject inventive cancer screening methodology include the use of blood serum from pregnant mammals of different species and embryonic blood serum used as the test serum, separately for the same patient evaluation, with non-pregnant serum of the same mammalian species as the control serum.

In the preferred embodiment, blood serum of a pregnant mare obtained preferably during the second trimester of pregnancy, is added to an aliquot of a patient's anticoagulated blood and in parallel, control serum from a non-pregnant horse is added to another aliquot of the patient's anticoagulated blood; the capillary tubes are positioned vertically or, in an alternative preferred embodiment, tilting at the top by about 45° away from the vertical, and a respective predetermined standardized period of time is allowed to elapse. The anticoagulant used for the preferred embodiment is lithium heparin. The ESR determination is then made for each capillary tube and the maximum ESR in the group is also established. Using these values, a presumptive cancer diagnosis is made according to the formula for a differential ESR derived cancer coefficient.

The subject inventive method of cancer screening is practiced as follows: 200 microliters of heparinized blood, using for example about 20 units of Lithium Heparin per 1 milliliter (“ml”) of blood, are put into each of the two ESR vials; in the case of plastic capillary tubes. About 50 microliters of the test serum is added to the first vial and 50 microliters of the control serum is added to the second vial with the patient's blood sample. Vials are shaken for less than 10 seconds, typically by inverting 4 to 5 times, and then the 50-mm glass capillary tubes are inserted into the vials so that blood-serum mixtures reach the same surface level in the capillary tubes; if the level is changed then the formula is changed to normalize the new distances.

As the standardized tube chosen to be used in practicing the present invention, capillary tubes from any of the many different manufacturers can be used. A glass 50-mm capillary tube with an inside diameter of 0.8 mm is used and preferably kept at 37° C. Lower temperatures, including room temperatures of 20° to 22° C., can be used but the reaction time is longer, as much as 1.5 hours; at 37° C. the time frame is 1 hour.

After about one hour the ESR value is determined, measured in millimeters. In order to increase the accuracy, more than one pair of capillary tubes is preferably used. When using more than one pair of capillary tubes, the arithmetic average of measurement results within each group—the test serum group and in the control serum group separately—is the value used to derive the cancer coefficient. Maximal ESR values among each patient's group of ESR tests are noted.

Establishing the difference in ESR measurements, or equivalently the differential ESR, in tubes with different sera and then multiplying by the maximum ESR determination measurement results and dividing the difference value by the normalization factor, in this case the normalization factor equals 50, to calculate the particular cancer coefficient. A cancer coefficient value greater than 1.5 is associated with a substantial probability about 85% that there is an ongoing malignancy in the patient; in general, higher cancer coefficients correlate with higher probabilities of ongoing and higher grade malignancies. Biopsy diagnosed cancer patients have been found with cancer coefficients reaching 4.0-5.0 and higher, which cancer coefficient values are associated with a much higher probability of on-going malignancy.

Different capillary tubes with different inside diameters can slightly change the measurements and therefore also change the calculated cancer coefficient significantly. For example, if as a capillary ESR system the SEDIPLAST® ESR System (Polymedco, Cortland, N.Y.) is used the time frame of the ESR measurement is changed to 30 to 40 minutes, and the cancer coefficient formula should be also changed. The new diagnostic cancer coefficient is calculated according to the following: differences in ESR measurements in tubes with different sera are divided by a normalization factor, which in this case equals 230, and is multiplied by the maximum of ESR measurement results. If the result of the cancer coefficient calculation is higher than 1.5 then an on-going cancer is presumptively diagnosed.

Other equivalent anticoagulants may be combined with whole blood, either manually or by obtaining commercially available collection tubes which contain an anticoagulant. The exemplary embodiments discussed below all were practiced using a green top collection tube with a 4.5 ml capacity and containing lithium heparin 72 units as an inside coating.

Sodium heparin is combined with whole blood with a stoichiometry in the range of about 12 to 20 units per ml of blood. Lithium heparin is used at about 13 to 20 units per ml of whole blood. Other anticoagulants used for this method step include without limitation:
Acid-Citrate-Dextrose (“ACD”);
ammonium heparin;
3.2% or 3.8% buffered tri-sodium citrate solution at 1 part citrate solution to 4 parts blood (VACUETTE® ESR tubes; Greiner Bio-one, Kremsmuenster, Austria); and so on.

The anticoagulant potassium ethylenediaminetetraacetic acid (“EDTA”) is considered to be unsuitable for the present ESR methodology.

It is within the contemplation and scope of the present subject invention that slightly different methods of ESR determination reactions can be used, for example, anticoagulants providing functional equivalence can be substituted for the preferred lithium heparin without any noticeable effect on the present cancer screening protocol; as another example, the test serum and the control serum may be loaded into blood collection tubes during their manufacture.

In order to improve its accuracy and reliability, it is believed to be within the contemplation and scope of the present subject invention for the present inventive cancer screening methodology to use two or more different sera for one patient. For example, the blood serum of a pregnant horse and the blood serum of a pregnant cow can be used separately as test sera. Such a screening test definitely has improved sensitivity and specificity. Other sera from different mammals can be used additionally during one screening test to further optimize specificity, efficiency and reliability.

The present subject inventive methodology further encompasses substantially equivalent cancer coefficient expressions using formulae that mathematically weight the difference between the test and control ESR determinations by multiplying it by the maximal ESR value in the tested group, normalizing the expression for the capillary tube dimensions; the equivalent cancer coefficients are then compared statistically with historical clinical data of cancer coefficients from patients with established cancers to provide a probability of an ongoing cancer in a patient.

The subject invention comprises a cancer screening methodology, as well as a diagnostic screening kit for performing the cancer screening methodology disclosed and claimed. The kit provides at least two kinds of serum—a first fluid designated ‘A’ referred to as the test serum, and a second fluid designated ‘C’ referred to as the neutral or control serum, which are to be combined with a patient's heparinized specimen. The sera can be provided for the test in a liquid, frozen or freeze-dried form and can further be provided in some commercially available vials.

The kit further comprises:
1) At least one test tube when using SEDIPLAST® (Polymedco, Cortland, N.Y.) plastic 100-mm capillary tubes or at least two test tubes when using glass 50-mm glass capillary tubes, preferably pre-loaded during manufacture with a predetermined effective amount of anticoagulant for the patient's blood specimen;
2) At least two standardized capillary tubes preferably the commercially available SEDIPLAST® ESR System plastic capillary tubes; other types of capillary tubes can be used, such as glass, but different volumes of blood will be needed.
3) At least two vials, equivalently referred to as cuvettes, for blood mixing, as are supplied with “SEDIPLAST®” capillary tubes;
   optionally included equipment comprises:
4) Laboratory pipettes with 40 to 500 microliter capability;
5) Test tube stands or other support for the capillary tubes.

The preferred embodiment of the subject invention contemplates that the methodology will be practiced in a facility equipped with a vibrator-mixer for blood serum mixing, and a thermostat-regulated ambient temperature about 37° C. that allows shorter test times.

### DESCRIPTION OF ILLUSTRATIVE EMBODIMENTS

Clinical examples of the present subject inventive method of a cancer screening diagnosis that has been put to practice in specific clinical cases. These 83 cases demonstrate how the subject inventive methodology can detect a wide variety of on-going cancers. These illustrative embodiments further emphasize the usefulness of the present subject cancer screening test for veterinary applications; the patient-subject who is tested for cancer may be human or some other mammal.

The following are clinical examples of the present subject inventive methodology of screening sixty human and twenty-three canine patients for an on-going malignancy. Each of the sixty clinical examples below was carried out by practicing the same method steps, the same embodiment of the present subject inventive methodology, namely:
Aliquots of about 3-4 mls of whole blood was obtained by venipuncture from each patient and transferred to a 4.5 ml green top blood collection tube containing 72 units of lithium heparin as the anticoagulant; brief shaking for a few seconds assures adequate mixing of the blood and anticoagulant.
About 400 microliters of patient blood was combined with about 50 microliters of pregnant horse serum in cuvette gently shaken by hand for about half a minute and then transferred to SEDIPLAST® (Polymedco, Cortland, N.Y.) plastic 100 mm capillary tube and left standing at room temperature of about 21° C. in the vertical condition for about 45 minutes; and
About 400 microliters of patient blood was combined with about 50 microliters of non-pregnant horse serum in a second SEDIPLAST® plastic 100 mm capillary tube, shaken by hand for about half a minute and left standing at about 21° C. in the vertical condition for about 45 minutes.
In each case about 400 microliters of patient blood was combined with about 50 microliters of serum. Measurements E₁, E₂ were taken of the height of the serum—erythrocyte sediment interface from the top of the meniscus in the capillary tube and recorded as the particular ESR value for that part of the cancer screening to be used in the cancer coefficient calculations.
The maximal ESR value E_{Max} for the group of ESR determinations were established for each patient.
Cancer coefficients for each patient's paired ESR tests (patient and control) are established according to the formula,
   K=(E₁−E₂)*E_{Max}/NF where the normalization factor NF=230.
   The following sixty examples involve human patients.
Example 1. A 49-year-old female was found to have uterine carcinoma and a cancer coefficient value of 1.8.
Example 2. This 44 year old female had a cancer coefficient value of 1.8 with tissue diagnosis of breast carcinoma.
Example 3. A female adult of unknown age presented with liver metastasis from an adenocarcinoma of unknown origin; her cancer coefficient was 3.6.
Example 4. A benign testicular tumor was found in this 29 year old male having a cancer coefficient value of zero.
Example 5. This 66 year old male with prostate cancer had a cancer coefficient value of 4.1.
Example 6. A cancer coefficient value of 3.8 was found for a 71 year old male with adenocarcinoma of the lung.
Example 7. A 30 year old female found to have uterine carcinoma with metastases had a cancer coefficient value of 4.1.
Example 8. A 43 year old female with breast cancer had a cancer coefficient value of 1.6.
Example 9. Prostate cancer in a 56 year old male was associated with a cancer coefficient value of 6.5.
Example 10. A 54 year old female with colon cancer and a uterine tumor presented with a cancer coefficient value of 4.3.
Example 11. This 58 year old female had a cancer coefficient value of 3.6 soon after resection of a bladder transitional cell carcinoma.
Example 12. An 86 year old female presented with a salivary gland cancer and had a cancer coefficient value of 1.9.
Example 13. A 66 year old female with lymphosarcoma of the head and neck had a cancer coefficient value of 4.8.
Example 14. Uterine carcinoma was associated in this 28 year old female with a cancer coefficient value of 7.9.
Example 15. A 73 year old female with lymphosarcoma had a cancer coefficient value of 9.5.
Example 16. A 53 year old female with histiocytic lymphocytic leukemia had a cancer coefficient value of 3.8.
Example 17. A 75 year old male with chronic lymphocytic leukemia had a cancer coefficient value of 3.7.
Example 18. This 56 year old female with gastric carcinoma had a cancer coefficient value of 2.3.
Example 19. Esophageal carcinoma in this 66 year old male was associated with a cancer coefficient value of 3.7.
Example 20. A right upper lobe lung cancer in this 43 year old male was associated with a cancer coefficient value of 2.3.
Example 21. A 30 year old female who was found to have lymphoma had a cancer coefficient value of 2.8.
Example 22. A 49 year old male with renal carcinoma had a cancer coefficient value of 10.1.
Example 23. This 27 year old female had a left ovarian teratocarcinoma and a cancer coefficient value of 3.6.
Example 24. A 74 year old female found to have colon carcinoma had a cancer coefficient value of 2.2.
Example 25. A 74 year old male with lymphosarcoma had a cancer coefficient value of 4.8.
Example 26. This 67 year old male found to have esophageal carcinoma had a cancer coefficient value of 6.2.
Example 27. A 62 year old male with lymphoma had a cancer coefficient value of 3.5.
Example 28. A 55 year old female found to have breast cancer had a cancer coefficient value of 1.4.
Example 29. Ovarian carcinoma was found in this 63 year old female who had a cancer coefficient value of 3.0.
Example 30. A 66 year old female found to have breast cancer had a cancer coefficient value of 1.4.
Example 31. A 56 year old female with colon carcinoma had a cancer coefficient value of 4.0.
Example 32. Gastric carcinoma in this 72 year old female was associated with a cancer coefficient value of 7.4.
Example 33. This 65 year old male found to have prostate carcinoma had a cancer coefficient value of 3.2.
Example 34. A 64 year old male with prostate carcinoma had a cancer coefficient value of 1.8.
Example 35. This 77 year old male with prostate carcinoma had a cancer coefficient value of 1.7.
Example 36. Lymphosarcoma in this 79 year old male was associated with a cancer coefficient value of 1.9.
Example 37. A 21 year old male diagnosed with Hodgkin's Lymphoma had a cancer coefficient value of 1.6.
Example 38. A 56 year old female with lymphoma had a cancer coefficient value of 2.8.
Example 39. Breast carcinoma in this 45 year old female was associated with a cancer coefficient value of 5.7.
Example 40. A 43 year old male with stage IIb breast cancer had a cancer coefficient value of 2.2.
Example 41. This 44 year old male with renal cell carcinoma metastatic to the liver had a cancer coefficient value of 5.5.
Example 42. A 30 year old patient with lymphoma had a cancer coefficient value of 4.8.
Example 43. A 24 year old male diagnosed with lung carcinoma had a cancer coefficient value of 2.7.
Example 44. A 76 year old male diagnosed with lung carcinoma had a cancer coefficient value of 3.1.
Example 45. Pancreatic carcinoma metastatic to the liver in this 67 year old male was associated with a cancer coefficient value of 4.8.
Example 46. A 75 year old female was diagnosed with lymphoma had a cancer coefficient value of 2.8.
Example 47. This 55 year old male with lymphosarcoma had a cancer coefficient value of zero.
Example 48. A 70 year old male diagnosed with lung cancer had a cancer coefficient value of 0.6.
Example 49. A 50 year old female with breast carcinoma had a cancer coefficient value of 6.5.
Example 50. Lymphoma in this 28 year old male was associated with a cancer coefficient value of 0.9.
Example 51. A dermato-fibrosarcoma diagnosed and surgically resected in this 44 year old female was associated with a post-operative cancer coefficient value of 1.5.
Example 52. A 67 year old female with a hepato-biliary carcinoma had a cancer coefficient value of 2.3.
Example 53. This 53 year old male diagnosed with esophageal carcinoma had a cancer coefficient value of 6.4.
Example 54. A 43 year old female with breast cancer had a cancer coefficient value of 2.2.
Example 55. A 59 year old female with lymphogranulomatosis had a cancer coefficient value of 1.4.
Example 56. Lymphoma in this 23 year old male was associated with a cancer coefficient value of zero.
Example 57. Lung carcinoma in this 46 year old male was associated with a cancer coefficient value of 5.1.
Example 58. Lymphogranulomatosis in this 31 year old female was associated with a cancer coefficient value of 4.5.
Example 59. A 36 year old female diagnosed with breast carcinoma had a cancer coefficient value of 5.6.
Example 60. A 27 year old female diagnosed with lymphosarcoma had a cancer coefficient value of 9.5.

The following 23 examples of the present subject cancer screening method was practiced on canine patients. Glass capillary tubes 50 mm long (Gus-Khrustalniy, Moscow, Russian Federation) were used in all of these exemplary embodiments; for the 50-mm glass capillary tubes the test and control aliquots of patient blood each comprised 200 microliters of patient blood; about 50 mls of pregnant and non-pregnant serums are added to respective patient whole blood aliquots. ESR determinations were taken after one hour. The cancer coefficient normalization factor equals 50 for these glass capillary tubes; all other method steps and elements were the same as practiced in the Examples 1-60.
Example 61. This healthy mixed-breed dog was found to have a cancer coefficient value of 0.2.
Example 62. An 8 year old Rottweiler with a diagnosis of endometritis but no malignancy had a cancer coefficient value of 0.2.
Example 63. A stray mixed breed dog having an ovarian malignancy had a cancer coefficient value of 1.8.
Example 64. A 9 year old Rottweiler having metastatic adenocarcinoma had a cancer coefficient value of 2.0.
Example 65. A 7 year old dog found to have malignant lesions in the spleen and liver had a cancer coefficient value of 6.0.
Example 66. This 9 year old poodle with breast cancer had a cancer coefficient value of 3.2.
Example 67. A mixed breed female dog with breast cancer had a cancer coefficient value of 3.0.
Example 68. A dog of unknown age found to have metastatic carcinoma with no apparent primary site had a cancer coefficient value of 4.9.
Example 69. Breast carcinoma was diagnosed in this 13 year old female dog having a cancer coefficient value of 0.8.
Example 70. A 10 year old female dog having breast carcinoma was found to have a cancer coefficient value of 0.6.
Example 71. This 13 year old dog with skin carcinoma had a cancer coefficient value of 4.5.
Example 72. A 3 year old dog having sarcoma with metastases had a cancer coefficient value of 1.7.
Example 73. A 10 year old female dog diagnosed with breast carcinoma had a cancer coefficient value of 4.0.
Example 74. Another 10 year old female dog diagnosed with breast carcinoma had a cancer coefficient value of 1.9.
Example 75. This 11 year old female poodle found to have breast carcinoma had a cancer coefficient value of 2.4.
Example 76. This 7 year old boxer was diagnosed with fibrosarcoma and had a cancer coefficient value of 4.8.
Example 77. A healthy non-pregnant 3 year old Rottweiler had a cancer coefficient value of 0.9.
Example 78. This 10 year old sheepdog had a cancer coefficient value of 0.9.
Example 79. A healthy non-pregnant 8-½ year old female dog had a cancer coefficient value of 1.3.
Example 80. A 2 year old Spaniel having eye cancer was found to have a cancer coefficient of 1.7.
Example 81. A healthy non-pregnant 3 year old Rottweiler without cancer had a cancer coefficient value of 1.3.
Example 82. This 5 year old female dog presenting with metastatic carcinoma had a cancer coefficient value of 2.8.
Example 83. A 5 year old dog with ear cancer had a cancer coefficient value of 2.2.
   For the clinical series of human patients described above the subject cancer screening test methodology demonstrated a sensitivity of 0.88, a specificity of 0.5, and a positive predictive value of 0.98.
   Exemplary Cancer Screening Test Procedure

Two new standardized cuvettes with corresponding capillary tubes are prepared; two small pipettes as supplied by SEDIPLAST® (Polymedco, Cortland, N.Y.) for blood delivery from patient into cuvettes. An anticoagulant such as heparin or sodium citrate must be added to patient blood, which must be obtained no more than 24 hours before the testing. If cuvettes and capillary tubes made by the manufacturers SEDIPLAST® (Polymedco, Cortland, N.Y.) are used then no more anticoagulant needs to be added since it has been added to the cuvettes during production. Cuvettes are labeled, such as #1 and #2 and so on.

Then, using the SEDIPLAST® equipment, 400 microliters of blood are taken from the green top tube, in which the whole blood is first put to combine with the heparin contained therein, and transferred into each cuvette: marked #1 and #2. Blood transfer to the cuvettes is done using standardized green top blood delivery tubes containing heparin, which are included with the standardized package of capillary tube equipment. Smaller tubes can be used to take blood from a finger stick. Then,
1. about 50 microliters of test serum (A) is added to blood in cuvette #1;
2. about 50 microliters of control serum (C) is added to blood in cuvette #2.
3. Each cuvette is shaken by hand or vibrated with a vibrator for about 5-10 seconds.
4. Capillary tubes are placed into each cuvette in a vertical orientation and mixed; the blood level meniscus should reach substantially the same height in the capillary tubes and be marked for subsequent reference.
5. After capillary tubes are set up the ESR process begins. The ESR process time is about 35-40 minutes when using SEDIPLAST® plastic 100 mm capillary tubes.
6. After the process time is up, a distance from each borderline between the transparent part of the blood mixture and the colored part of the blood mixture and the meniscus is measured using the scale on the capillary tubes if glass tubes are used or other metric scale for plastic tubes. Two ESR determination values are measured, the maximum ESR “E_{Max}” and the minimum ESR, “E_{Min}” for both capillary tube groupings.
7. If there is no significant difference in ESR borderline heights for A and for C, then the cancer coefficient value from the test will be no greater than 1.5 and the presumptive conclusion is that there is no on-going cancer.
8. If there is a significant difference between the sedimentation heights, which is to say the difference between the ESR determinations, which difference yields a cancer coefficient greater than 1.5, then the result of the test is deemed positive, and the presumptive conclusion is that there is an on-going cancer.
   When 50 mm glass capillary tubes (Gus-Khrustalniy, Moscow, Russian Federation) are used the formula of the cancer coefficient is:
   K=[(E_{Max}−E_{Min})*E_{Max}]/NF where NF=50 in this case.
   When the SEDIPLAST® ESR System (Polymedco, Cortland, N.Y.) 100 mm capillary tubes are used the formula of the cancer coefficient is:
   K=(E_{Max}−E_{Min})*E_{Max}/NF where NF=230 when SEDIPLAST® plastic 100-mm capillary tubes are used.
   A cancer coefficient value of 1.5 is considered the high normal value of the cancer coefficient for non-malignant situations. Cancers typically give higher coefficients, as high as 6-8, and even higher. The probability of a more aggressively malignant cancer is proportionately higher with higher cancer coefficient values. The subject Inventors are of the opinion that higher cancer coefficient values correlate with the presence of metastatic disease and indicate a more virulent and higher grade malignancy.

When freeze-dried sera are used, the serum is reconstituted to the original concentration using distilled water 1:100 weight/weight; for each 10 grams of freeze-dried sera about one liter, or equivalently, 1000 mls of water would be used. However, if during manufacturing the freeze-dried sera is added to the cuvettes then additional water should not be added for the practice of the subject invention's methodology.

Although this invention has been described in connection with specific forms and embodiments thereof, it should be appreciated that various modifications other than those discussed above may be resorted to without departing from the spirit or scope of the invention. For example, equivalent elements may be substituted for those specifically shown and described, certain features may be used independently of other features, and in certain cases, particular locations of elements may be reversed or interposed, all without departing from the spirit or scope of the invention as defined in the appended claims.

We claim:

1. A method of cancer detection in a patient, comprising the steps of:
(a) providing a pre-determined quantity of a test serum chosen from the group consisting of serum from a pregnant mammal and fetal embryonic serum from a pre-determined species respectively;
(b) providing a pre-determined quantity of a control serum from a second mammal belonging to said pre-determined species, wherein said second mammal is not pregnant;
(c) providing a predetermined quantity of whole blood of said patient;
(d) combining in pre-determined proportions and conditions said test serum with said whole blood of said patient and establishing a first Erythrocyte Sedimentation Rate (“ESR₁”) according to a standardized technique;
(e) combining in pre-determined proportions and conditions said control serum with said whole blood of said patient and establishing a second Erythrocyte Sedimentation Rate (“ESR₂”) according to said standardized technique;
(f) establishing a maximal measured ESR (“ESRₘₐₓ×”);
(g) determining a normalization factor (“NF”) proportional to a dimension of a capillary tube used in said standardized technique; and,
(h) determining a cancer coefficient equal to an absolute value of
[(ESR₁−ESR₂)*ESRₘₐₓ]/NF;
wherein said cancer coefficient corresponds and correlates with a predetermined probability of an on-going malignant process when said cancer coefficient is greater than a respectively predetermined normalized cancer coefficient value.

2. The method of cancer detection in a patient as recited in claim 1 wherein said test serum comprises a serum from said pregnant mammal obtained at a predetermined gestational time.

3. The method of cancer detection in a patient as recited in claim 2 wherein said test serum comprises a serum from a pregnant mammal belonging to said pre-determined species during a second trimester.

4. The method of cancer detection in a patient as recited in claim 1 wherein said standardized technique comprises a tilted condition for said capillary tubes in a range of about 10° to about 55° away from a vertical condition.

5. The method of cancer detection in a patient as recited in claim 1 wherein said standardized technique further comprises a 45° tilted condition from a vertical condition for said capillary tubes.

6. The method of cancer detection in a patient as recited in claim 1 wherein said standardized technique further comprises a temperature in the range of about 20° to about 37° C. in which to practice said method.

7. The method of cancer detection in a patient as recited in claim 1 with said cancer being a malignant neoplastic disease entity chosen from the group of malignant neoplastic disease entities consisting of adenocarcinomas, lymphomas, multiple myelomas, prostate carcinomas, transitional cell bladder carcinomas, squamous cell carcinomas, sarcomas, malignant teratocarcinomas, thyroid carcinomas, pancreatic carcinomas, lung carcinomas, cervical carcinomas, ovarian carcinomas, breast carcinomas, endocrine carcinomas, colon carcinomas, malignant melanomas, testicular cancers, leukemias, gastrointestinal carcinomas, head and neck carcinomas, carcinomas of unknown origin, and combinations thereof.

8. A new use for fetal embryonic serum of a mammal for detecting a malignancy in a patient, comprising:
(a) providing a pre-determined quantity of fetal embryonic serum belonging to a pre-determined species;
(b) providing a pre-determined quantity of a control serum from a mammal belonging to said pre-determined species, wherein said mammal is not pregnant;
(c) providing a predetermined quantity of whole blood of said patient;
(d) combining in pre-determined proportions and conditions said fetal embryonic serum with said whole blood of said patient and establishing a first Erythrocyte Sedimentation Rate (“ESR₁”) according to a standardized technique;
(f) combining in pre-determined proportions and conditions said non-pregnant mammal serum with said whole blood of said patient and establishing a second Erythrocyte Sedimentation Rate (“ESR₂”) according to said standardized technique;
(g) establishing a maximal measured ESR (“ESRₘₐₓ”);
(h) determining a normalization factor (“NF”) proportional to a dimension of a capillary tube used in said standardized technique; and,
(i) determining a cancer coefficient equal to an absolute value of
[(ESR₁−ESR₂)*ESRₘₐₓ]/NF;
wherein said cancer coefficient corresponds and correlates with a predetermined probability of an on-going malignant process when said cancer coefficient is greater than a respectively predetermined normalized cancer coefficient value.

9. A new use for an erythrocyte sedimentation rate test, comprising the steps of:
(a) providing a pre-determined quantity of a test serum chosen from the group consisting of serum from a pregnant mammal and fetal embryonic serum from a pre-determined species respectively;
(b) acquiring a predetermined quantity of whole blood of said patient;
(c) combining in pre-determined proportions and conditions said test serum with said whole blood of said patient and establishing a first Erythrocyte Sedimentation Rate (“ESR₁”) according to a standardized technique;
(d) determining a normalization factor (“NF”) proportional to at least one dimension of a capillary tube used in said standardized technique;
(j) providing a pre-determined quantity of serum from a non-pregnant mammal belonging to said pre-determined species;
(k) combining in pre-determined proportions and conditions said non-pregnant mammal serum with said whole blood of said patient and establishing a second Erythrocyte Sedimentation Rate (“ESR₂”) according to said standardized technique;
(l) establishing a maximal measured ESR (“ESRₘₐₓ”); and,
(m) determining a cancer coefficient equal to an absolute value of
[(ESR₁−ESR₂)*ESRₘₐₓ]/NF;
wherein said cancer coefficient corresponds and correlates with a predetermined probability of an on-going malignant process in said patient when said cancer coefficient is greater than a respectively predetermined normalized cancer coefficient value.

10. The new use for an erythrocyte sedimentation rate test as recited in claim 9 wherein said standardized technique comprises a tilted condition for said capillary tubes in a range of about 10° to about 55° away from a vertical condition.

11. The new use for an erythrocyte sedimentation rate test as recited in claim 9 wherein said standardized technique comprises a 45° tilted condition away from a vertical condition for said capillary tubes.

12. The new use for an erythrocyte sedimentation rate test as recited in claim 9 wherein said standardized technique further comprises a temperature in the range of about 20° to about 37° C. in which to practice said method.

13. The new use for an erythrocyte sedimentation rate test as recited in claim 9 wherein said test serum comprises a serum from said pregnant mammal obtained at a predetermined gestational time 14. The new use for an erythrocyte sedimentation rate test as recited in claim 13 wherein said predetermined gestational time of said pregnant mammal is in a second trimester.
